(19) United States
(12) Patent Application Publication
(10) Pub. No.: US 2009/0304786 A1
(43) Pub. Date: Dec. 10, 2009

(54) Stable Dosage Forms of an Antidepressant

(76) Inventors: Viswaprasad Varanasi, Andhra Pradesh (IN); Haranatha Babu Balanagu, Andhra Pradesh (IN); Hidaytulla Shamshuddin Aga, Andhra Pradesh (IN); Kishor Dattatray Deo, Andhra Pradesh (IN); Umesh Nandkumar Khatavkar, Andhra Pradesh (IN); Sivakumaran Meenakahisunderam, Andhra Pradesh (IN)

Correspondence Address: Jay Akhave, 2058 N Mills Ave, #612, Claremont, CA, 91711, US

(21) Appl. No.: 12/085,534

(22) PCT Filed: Nov. 23, 2006
(86) PCT No.: PCT/IB2006/003409

§ 371 Date: Aug. 10, 2009

(30) Foreign Application Priority Data
Nov. 25, 2005 (IN) 1731/CHE/2005

(51) Int. Cl. A61K 9/48; A61K 31/138; A61K 9/28; A61K 9/20; A61K 9/22; A61P 25/24

(52) U.S. Cl. 424/452; 514/646; 424/465; 424/468

(57) ABSTRACT

The present invention relates to stable solid dosage forms of an antidepressant. More particularly, the present invention relates to stable solid dosage forms of bupropion hydrochloride. The present invention also relates to a process for the preparation of stable solid dosage forms of bupropion hydrochloride.

### FIELD OF THE INVENTION

The present invention relates to stable solid dosage forms of an antidepressant. More particularly, the present invention relates to stable solid dosage forms of bupropion hydrochloride.

The present invention also relates to a process for the preparation of stable solid dosage forms of bupropion hydrochloride.

### BACKGROUND OF THE INVENTION

Bupropion hydrochloride is an antidepressant of the aminoketone class and a non-nicotine aid to smoking cessation; is chemically unrelated to tricyclic, tetracyclic, selective serotonin re-uptake inhibitor, or other known antidepressants.

Bupropion, described in U.S. Pat. Nos. 3,819,706 and 3,885,046, is currently available as the hydrochloride salt and chemically it is (±)-1-(3-chlorophenyl)-2-[(1,1-dimethylethyl)amino]-1-propanone hydrochloride.

Bupropion hydrochloride is commercially available in the US as immediate release tablets under the trade name Wellbutrin®, extended release tablets under the trade name Wellbutrin XL® and sustained release tablets under the trade name Wellbutrin SR® & Zyban®.

Bupropion hydrochloride is a water-soluble, crystalline solid, highly hygroscopic and susceptible to decomposition. The stability of bupropion hydrochloride may be affected by factors including formulation and storage conditions. Because of the drug's instability, the shelf life of bupropion formulations have proven to be problematic. Many formulations have been reported for improving the storage stability of bupropion hydrochloride. Following are some of patents/publications, which discloses stable bupropion hydrochloride compositions:

U.S. Pat. No. 5,541,231 discloses that the instant release tablet form sold under the brand name Wellbutrin® are quite suitable for the indicated use, the method of manufacturing these is less than desirable based on cost as well as process conditions. To overcome stability problems and to protect bupropion from degradation, U.S. Pat. No. '231, U.S. Pat. No. 5,358,970, U.S. Pat. No. 5,763,493 discloses stable solid dosage form of bupropion, using stabilizers such as absorbic acid or isoascorbic acid, L-cysteine hydrochloride, glycine hydrochloride, malic acid, sodium metabisulfite, citric acid, tartaric acid and L-cystine dihydrochloride. The compositions prepared as per this patent, when stored for 6 weeks at about 50° C. and at about 27% relative humidity contain at least about 80% of the labelled amount of bupropion hydrochloride.

U.S. Pat. No. 5,731,000 discloses a solid composition comprising bupropion hydrochloride and a pharmaceutically acceptable stabilizer, wherein stabilizer is selected from an organic acid, a carboxylic acid, an acid salt of an amino acid and sodium metabisulphite.

U.S. Pat. No. 5,968,553 discloses a solid composition comprising bupropion hydrochloride and a pharmaceutically acceptable stabilizer, wherein an inorganic acid is used as stabilizer.

U.S. Pat. Nos. 6,143,327, 6,096,341 disclose delayed release coated tablet of bupropion hydrochloride free of stabilizer and free of pore-forming agent.

U.S. Pat. No. 6,153,223 discloses a method of preparing stable composition comprising premixing a pharmaceutically acceptable carrier and an aqueous solution of an acid selected from hydrochloric acid, nitric acid, sulfuric acid, and phosphoric acid; drying the premixture and admixing dried premixture with a pharmaceutical agent unstable at a pH above about 3.5, wherein the acidic pharmaceutically acceptable carrier provides an acidic stabilizing microenvironment around the pharmaceutical agent.

U.S. Pat. No. 6,194,002 discloses a solid composition comprising bupropion hydrochloride and fumaric acid, wherein the ratio of fumaric acid to bupropion hydrochloride by weight is from about 0.05 to about 2.0.

U.S. Pat. No. 6,238,697 discloses method of preparing bupropion hydrochloride tablets by direct compression without employing a slugging or wet granulation process.

U.S. Pat. Nos. 6,242,496 and 6,221,917 disclose a solid composition comprising bupropion hydrochloride and a pharmaceutically acceptable stabilizer, wherein dicarboxylic acid is used as stabilizer.

U.S. Pat. No. 6,033,686 discloses controlled release tablet, free of stabilizer and free of pore-forming agent comprising a core of bupropion hydrochloride, a binder and a lubricant and a coating consisting essentially of a water-insoluble, water-permeable film-forming polymer, a plasticizer and a water-soluble polymer.

U.S. Pat. No. 6,306,436 discloses methods of stabilizing bupropion hydrochloride in a sustained release formulation without incorporating added acid in the formulation, comprising coating crystals of bupropion hydrochloride with a cellulosic polymer and a pharmaceutically acceptable carrier to provide sustained release of the bupropion hydrochloride.

U.S. Pat. No. 6,333,332 discloses pharmaceutical composition comprising bupropion hydrochloride and a pharmaceutically acceptable stabilizer, wherein stabilizer is selected from the group consisting of an organic base and a salt of an inorganic acid.

U.S. Pat. No. 6,462,237 discloses inclusion complex of bupropion hydrochloride and beta-cyclodextrin.

U.S. Pat. No. 6,482,987 discloses method of preparing a stable composition of bupropion hydrochloride in solid form comprising dry blending bupropion hydrochloride and solid stabilizer; dry milling the blended material; and preparing a solid dosage form.

U.S. Pat. No. 6,652,882 discloses a controlled release pharmaceutical formulation comprising of bupropion hydrochloride, 20% to about 25% by weight of an uncrosslinked polymer and 1% to about 70% by weight of a crosslinked insoluble polymer.

U.S. Pat. No. 6,893,660 discloses a controlled release pharmaceutical preparation of bupropion in which the degradation of active ingredient is prevented without the use of a stabilizer.

U.S. Pat. No. 6,589,553 discloses a controlled release bupropion formulation comprising a pH dependent coating of hydroxypropyl methylcellulose phthalate.

US 2003/0044462 discloses a solid composition comprising bupropion hydrochloride and carboxyvinyl polymer. It is also disclosed that the main degradation product of bupropion hydrochloride is 3-chlorobenzoic acid.

US 2005/0112198 discloses compositions of buproprion hydrochloride comprising at least one member of the group consisting of butylated hydroxyanisole, butylated hydroxytoluene and an ion exchange resin.

US 2006/0204571 discloses a stable oral pharmaceutical composition comprising bupropion or its pharmaceutical acceptable salt, intimately blending with one or more compatible excipient selected from talc and potassium chloride where total impurities are present in amounts from 0% to not more than 3.3% w/w of the bupropion hydrochloride.

WO 00/030685 discloses solid composition comprising bupropion hydrochloride and sodium bisulfate as stabilizer.

WO 03/086362 discloses stable bupropion hydrochloride tablet, wherein the tablet is free of stabilizer.

WO 04/045584 discloses solid composition comprising bupropion hydrochloride and stabilizer, wherein the stabilizer comprises glucono delta lactone or its corresponding open chain hydroxy acid derivative.

The above prior art references disclose stable bupropion hydrochloride compositions using different types of stabilizers such as amino acids, organic acid, inorganic acid, fumaric acid or dicarboxylic acid, organic base, carboxyvinyl polymer, glucono delta lactone, sodium metabisulfite and carboxyvinyl polymer. Few of the prior art references disclose stable compositions of bupropion hydrochloride by coating the drug and separating excipients from direct contact with drug. However, still there is a need for preparing stable solid dosage form of bupropion hydrochloride, which involves simple and cost effective process. The inventors of the present invention during their continuous effort to develop stable dosage forms of bupropion hydrochloride found that the use of phthalates as a stabilizer provides dosage forms that are stable even after storage for longer period at accelerated conditions. Phthalates are known as acid resistant coating material for delaying the release of the active ingredient from the dosage form and commonly used in the delayed release dosage forms. However, the inventors of the present invention surprisingly found that phthalates when used in low quantity acts as stabilizer and minimize the degradation of bupropion hydrochloride.

### OBJECTIVE OF THE INVENTION

Accordingly, the main objective of the present invention is to provide stable solid dosage forms of bupropion hydrochloride.

Yet, another embodiment of the present invention is to provide simple, cost effective and efficient process for preparing stable solid dosage forms of bupropion hydrochloride.

Yet, another objective of the present invention is to provide stable solid dosage forms of bupropion hydrochloride in such a way that it will comply with the reference product in terms of in vitro parameters like dissolution, disintegration, etc and in vivo parameters like bioequivalence.

### SUMMARY OF THE INVENTION

Accordingly, the present invention provides stable solid dosage forms comprising bupropion hydrochloride and an effective stabilizing amount of hydroxypropyl methylcellulose phthalate.

The invention also provides a process for preparing stable bupropion hydrochloride dosage form comprising granulating bupropion hydrochloride and an effective stabilizing amount of hydroxypropyl methylcellulose phthalate and one or more pharmaceutically acceptable excipients, lubricating the granules and finally filling the granules into capsules or compressing into tablets.

### DETAILED DESCRIPTION OF THE INVENTION

In an embodiment of the present invention, bupropion hydrochloride having particle size wherein 90% of the particles of size from about 100 to about 500 μm is used.

The stable solid dosage form of bupropion hydrochloride of the present invention, further comprise one or more pharmaceutically acceptable excipients selected from the group consisting of fillers, disintegrants, binders, lubricants, polymers and the like.

The amount of hydroxypropyl methylcellulose phthalate used as stabilizer according to the present invention confers an excellent stability to the dosage form even after storage for longer periods at accelerated conditions.

In yet another embodiment of the present invention, the stable dosage forms retain at least 80% of its potency and preferably at least 90% after one year of storage at room temperature and 35-60% humidity. For example if the dosage form initially contains 100 mg bupropion hydrochloride (labeled amount) at the time of preparation, after one-year of storage at least 80 mg of bupropion hydrochloride will remain in the tablet.

In yet another embodiment of the present invention, the stable dosage forms contain the active ingredient and excipients, in weight to weight percentages of about 10% to about 50% of bupropion hydrochloride, about 0.25% to about 10% of stabilizer, about 40% to about 90% of diluent, about 1% to about 5% of disintegrant and about 0.5% to about 5% of lubricant.

Suitable fillers used according to the present invention are selected from microcrystalline cellulose, lactose, starch, pregelatinized starch, modified starch, dextrose, sucrose, mannitol and sorbitol or a combination thereof.

Suitable disintegrants used according to the present invention are selected from low substituted hydroxypropyl cellulose, croscarmellose sodium, sodium starch glycolate, crospovidone, pregelatinized starch, and sodium carboxymethyl cellulose or a combination thereof.

Suitable binders of the invention are selected from hydroxypropyl cellulose, hydroxypropyl methylcellulose, polyvinylpyrrolidone, starch, pregelatinized starch and the like.

Suitable lubricants used according to the present invention are selected from stearic acid, magnesium stearate, calcium stearate, zinc stearate, talc, vegetable oil, and sodium stearyl fumarate or a combination thereof.

In an embodiment of the present invention, the dosage form of bupropion hydrochloride may be in the form of granules, pellets, capsules, conventional tablets, sustained release tablets, controlled release tablets or extended release tablets. The sustained release or extended release tablets further comprises various release retrading polymer selected from hydroxypropyl methylcellulose, hydroxypropyl cellulose, hydroxyethyl cellulose, ethyl cellulose, xanthan gum, alginic acid or its salt, methacrylic acid derivatives, polyethylene oxide or a combination thereof.

The different formulation processes that can be employed for making the dosage forms are dry granulation (slugging or compaction), wet granulation, and direct compression. Preferably, the dosage forms of the present invention are prepared by wet granulation technique.

In yet another embodiment of the invention, the wet granulation process for the preparation of stable solid dosage form of bupropion hydrochloride comprises the following steps:

i) preparing granules of bupropion hydrochloride, an effective amount of stabilizer and excipients using solvent,
ii) drying the wet granules obtained in step (i) and blending with extragranular excipients,
iii) lubricating the blend obtained in step (ii),
iv) compressing the lubricated blend of step (iii) into tablets or filled into capsules and
v) optionally coating the tablets with film forming agents.

Suitable solvents used for preparing stable bupropion hydrochloride dosage forms include water, ethanol, acetone, methylene chloride, methanol, and isopropanol or a combination thereof.

The compositions are normally dried to a sufficient extent, so that the total content of added water remaining is 3.0% wt or less. Even though a wet granulation process is used, the dosage form of the present invention exhibits an excellent stability.

In yet another embodiment of the present invention, there is provided a method for treating depression by administering stable dosage forms of bupropion hydrochloride of the present invention to a patient in need thereof.

The solid dosage forms of bupropion hydrochloride of the present invention may be packed in HDPE container with molecular sieve sachets or the container is purged with nitrogen gas in order to provide oxygen free environment.

The following examples further exemplify the invention and are not intended to limit the scope of the invention. It is obvious to those skilled in the art to find out the composition for other dosage forms and substitute the equivalent excipients as described in this specification or with the one known to the industry.

The manufacturing process for the preparation of stable tablet dosage forms of bupropion hydrochloride described in examples 1-3 is as given below:

a) bupropion hydrochloride and microcrystalline cellulose were sifted and mixed in a granulator,
b) binder solution of hydroxypropyl methylcellulose phthalate in a mixture of acetone and ethanol was prepared,
c) granulated the dry mix obtained in step (a) with a binder solution of step (b),
d) dried the granules obtained in step (c) and blended with microcrystalline cellulose and low substituted hydroxypropyl cellulose,
e) lubricated the blend of step (d) with stearic acid,
f) compressed the lubricated blend obtained in step (e) and
g) coated the tablets obtained in step (f) with opadry.

### Example 1

| S. No. | Ingredients | Qty/tab (mg) |
|---|---|---|
| | Intragranular portion | |
| 1 | Bupropion Hydrochloride | 100.00 |
| 2 | Microcrystalline cellulose | 78.00 |
| 3 | Hydroxypropyl methylcellulose Phthalate | 10.00 |
| 4 | Acetone | Qs |
| 5 | Ethanol | Qs |
| | Extragranular portion | |
| 1 | Microcrystalline cellulose | 100.00 |
| 2 | Low substituted Hydroxypropyl cellulose | 10.00 |
| 3 | Stearic acid | 2.00 |

### Example 2

| S. No. | Ingredients | Qty/tab (mg) |
|---|---|---|
| | Intragranular potion | |
| 1 | Bupropion Hydrochloride | 100.00 |
| 2 | Microcrystalline cellulose | 200.0 |
| 3 | Hydroxypropyl methylcellulose Phthalate | 5.00 |
| 4 | Acetone | Qs |
| 5 | Ethanol | Qs |
| | Extragranular portion | |
| 1 | Low substituted hydroxypropyl cellulose | 10.00 |
| 2 | Microcrystalline cellulose | 298.00 |
| 3 | Stearic acid | 7.00 |

### Example 3

| S. No. | Ingredients | Qty/tab (mg) |
|---|---|---|
| | Intragranular portion | |
| 1 | Bupropion Hydrochloride | 100.00 |
| 2 | Hydroxypropyl methylcellulose Phthalate | 2.00 |
| 3 | Acetone | Qs |
| 4 | Ethanol | Qs |
| | Extragranular portion | |
| 1 | Low substituted hydroxypropyl cellulose | 10.00 |
| 2 | Microcrystalline cellulose | 100.00 |
| 3 | Stearic acid | 2.00 |

The manufacturing process for the preparation of stable tablet dosage forms of bupropion hydrochloride described in examples 4-6 is as given below:

a) bupropion hydrochloride, hydroxypropyl methylcellulose phthalate and intragranular microcrystalline cellulose were sifted and mixed in a granulator,
b) granulated the dry mix obtained in step (a) with water,
c) dried the granules obtained in step (b) and blended with extragranular microcrystalline cellulose and low substituted hydroxypropyl cellulose,
d) lubricated the blend of step (c) with stearic acid,
e) compressed the lubricated blend obtained in step (d) and
f) coated the tablets obtained in step (e) with opadry.

### Example 4

| S. No. | Ingredients | Qty/tab (mg) |
|---|---|---|
| | Intragranular portion | |
| 1 | Bupropion Hydrochloride | 100.00 |
| 2 | Microcrystalline cellulose | 200.0 |
| 3 | Hydroxypropyl methylcellulose Phthalate | 5.00 |
| 4 | Purified water | Qs |
| | Extragranular portion | |
| 1 | Low substituted hydroxypropyl cellulose | 10.00 |
| 2 | Microcrystalline cellulose | 298.00 |
| 3 | Stearic acid | 7.00 |

### Example 5

| S. No. | Ingredients | Qty/tab (mg) |
|---|---|---|
| | Intragranular portion | |
| 1 | Bupropion Hydrochloride | 100.00 |
| 2 | Hydroxypropyl methylcellulose Phthalate | 2.00 |
| 3 | Purified water | Qs |
| | Extragranular portion | |
| 1 | Low substituted hydroxypropyl cellulose | 10.00 |
| 2 | Microcrystalline cellulose | 100.00 |
| 3 | Stearic acid | 2.00 |

### Example 6

| S. No. | Ingredients | Qty/tab (mg) |
|---|---|---|
| | Intragranular portion | |
| 1 | Bupropion Hydrochloride | 100.00 |
| 2 | Microcrystalline cellulose | 200.0 |
| 3 | Hydroxypropyl methylcellulose Phthalate | 5.00 |
| 4 | Purified water | Qs |
| 5 | Isopropyl Alcohol | Qs |
| | Extragranular portion | |
| 1 | Low substituted hydroxypropyl cellulose | 10.00 |
| 2 | Microcrystalline cellulose | 298.00 |
| 3 | Stearic acid | 7.00 |

### Example 7

| S. No. | Ingredients | Qty/tab (mg) |
|---|---|---|
| | Intragranular portion | |
| 1 | Bupropion Hydrochloride | 100.0 |
| 2 | Microcrystalline Cellulose | 254.0 |
| 3 | Hydroxypropylmethylcellulose phthalate | 4.0 |
| 4 | Hydroxypropylmethylcellulose | 2.0 |
| 5 | Ethanol | QS |
| | Extragranular portion | |
| 1 | Microcrystalline Cellulose | 251 |
| 2 | Low substituted hydroxypropylcellulose | 2.0 |
| 3 | Stearic Acid | 7.0 |

The manufacturing process for the preparation of stable tablet dosage forms of bupropion hydrochloride described in examples 7 is as given below:

a) bupropion hydrochloride, microcrystalline cellulose hydroxypropylmethylcellulolse phthalate, hydroxypropyl methylcellulose were shifted,
b) granulated the content obtained in step (a) with ethanol,
c) dried the granulated content obtained in step (b),
d) blended the shifted granules obtained with step (c) with extragranular microcrystalline cellulose and low substituted hydroxypropylcellulose,
e) lubricated the blend of step (d) with stearic acid,
f) compressed the lubricated blend of step (e) into tablets and
g) coated the tablets obtained in step (f) with Opadry.

### Example 8

| S. No. | Ingredients | Qty/tab (mg) |
|---|---|---|
| | Intragranular portion | |
| 1 | Bupropion Hydrochloride | 100.0 |
| 2 | Microcrystalline Cellulose | 254.0 |
| 3 | Hydroxypropylmethylcellulose phthalate | 4.0 |
| 4 | Hydroxypropylmethylcellulose | 2.0 |
| 5 | Isopropyl Alcohol | QS |
| 6 | Purified Water | QS |
| | Extragranular portion | |
| 1 | Microcrystalline Cellulose | 251.0 |
| 2 | Low substituted hydroxypropylcellulose | 2.0 |
| 3 | Stearic Acid | 7.0 |

The stable solid dosage form of bupropion hydrochloride described in example 8 was prepared by the similar procedure followed in example 7.

Bupropion hydrochloride tablets prepared according the present invention were found to be stable. The stability data obtained for 3 months at 40° C., 75% relative humidity is shown in table 1.

TABLE 1

Assay

| Example No. | Initial | 3M |
|---|---|---|
| 7 | 100.3 | 99.9 |
| 8 | 102.53 | 102.7 |

We claim:

1. A stable solid dosage form comprising bupropion hydrochloride and an effective stabilizing amount of hydroxypropyl methylcellulose phthalate.

2. The stable solid dosage form as claimed in claim 1, wherein the stabilizing amount of hydroxypropyl methylcellulose phthalate is in the range of 0.5% to about 10% w/w of the composition.

3. The stable solid dosage form as claimed in claim 1, further comprises one or more pharmaceutically acceptable excipients such as fillers, disintegrants, binders, lubricant and polymers.

4. The stable solid dosage form as claimed in claim 3, wherein the filler is selected from the group consisting of microcrystalline cellulose, lactose, starch, pregelatinized starch, modified starch, dextrose, sucrose, mannitol, and sorbitol or a combination thereof.

5. The stable solid dosage form as claimed in claim 3, wherein the disintegrant is selected from the group of low substituted hydroxypropyl cellulose, croscarmellose sodium, sodium starch glycolate, crospovidone, pregelatinized starch, and sodium carboxymethyl cellulose or a combination thereof.

6. The stable solid dosage form as: claimed in claim 3, wherein the binder is selected from hydroxypropyl cellulose, hydroxypropyl methylcellulose, polyvinylpyrrolidone, starch and pregelatinized starch.

7. The stable solid dosage form as claimed in claim 3, wherein the lubricant is selected from stearic acid, magnesium stearate, calcium stearate, zinc stearate, talc, vegetable oil, and sodium stearyl fumarate or a combination thereof.

8. The stable solid dosage form as claimed in claim 1, is in the form of granules, capsule, conventional tablet, sustained release tablet, controlled release tablet or extended release tablet.

9. The stable solid dosage form as claimed in claim 3, wherein the polymer is selected from hydroxypropyl methylcellulose, hydroxypropyl cellulose, hydroxyethyl cellulose, ethyl cellulose, xantum gum, alginic acid or its salt, methacrylic acid derivatives and polyethylene oxide or a combination thereof.

10. A process for the preparation of stable solid dosage form comprising bupropion hydrochloride and an effective stabilizing amount of hydroxypropyl methylcellulose phthalate, which comprises the steps of
(i) preparing granules of bupropion hydrochloride, an effective stabilizing amount of hydroxypropyl methylcellulose phthalate and excipients using solvent,
(ii) drying the wet granules obtained in step (i) and blending with extragranular excipients,
(iii) lubricating the blend obtained in step (ii),
(iv) compressing the lubricated blend of step (iii) into tablets or filled into capsules and
(v) optionally coating the tablets with film forming agents or with sustained release or controlled release or extended release polymers.
